(19) United States
(12) Patent Application Publication
(10) Pub. No.: US 2015/0184144 A1
(43) Pub. Date: Jul. 2, 2015

(54) Liquid Enzyme Composition and Method for Enzyme Recovery

(71) Applicant: Novozymes A/S, Bagsvaerd (DK)

(72) Inventors: Roar Hauch, Frederiksberg (DK); Helene Munthe Jensen, Slagelse (DK); Ole Simonsen, Soeborg (DK)

(21) Appl. No.: 14/409,872

(22) PCT Filed: Jul. 4, 2013
(86) PCT No.: PCT/EP2013/064144

§ 371 Date: Dec. 19, 2014

Related U.S. Application Data
(60) Provisional application No. 61/692,449, filed on Aug. 23, 2012.
(60) Provisional application No. 61/669,347, filed on Jul. 9, 2012.

(30) Foreign Application Priority Data
Jul. 6, 2012 (EP) 12175293.5
Aug. 23, 2012 (EP) 12181490.9

(51) Int. Cl. C12N 9/96; A01N 37/10; A01N 37/06; A01N 37/02

(52) U.S. Cl. CPC C12N 9/96; A01N 37/02; A01N 37/10; A01N 37/06

(57) ABSTRACT

The addition of a a C₆-C₁₂ fatty acid or a salt thereof has an antimicrobial effect on Lactobacillus in a liquid enzyme composition.

### FIELD OF THE INVENTION

The present invention relates to a liquid enzyme composition. It also relates to a method of recovering and/or purifying an enzyme from a culture broth.

### BACKGROUND OF THE INVENTION

Liquid enzyme products are used commercially in large quantities as processing aids in various production processes, e.g. in the production of bioethanol from starch or (hemi)cellulosic raw materials. The enzyme products are generally produced by cultivation of microorganisms, followed by recovery, purification, standardization and formulation of the culture broth.

Growth of microorganisms in the liquid enzyme composition during recovery, purification, storage and transportation is a concern. Both bacteria, yeast and mold may cause problems, and particularly the growth of Lactobacillus tends to be problematic. To restrain the growth of such microorganisms, the solids content is commonly kept high in the final product, and the growth of microorganisms can be further inhibited, e.g. by adding antimicrobial agents such as sulfite, sorbate and benzoate.

### SUMMARY OF THE INVENTION

The inventors have found that the addition of a C₆-C₁₂ fatty acid or a salt thereof has an antimicrobial effect on Lactobacillus in a liquid enzyme composition. Accordingly, the invention provides a liquid composition, which comprises an enzyme and a C₆-C₁₂ fatty acid or a salt thereof in an amount which has an antimicrobial effect on Lactobacillus.

The invention also provides a method of recovering, purifying and/or formulating an enzyme from a culture broth, comprising adding a C₆-C₁₂ fatty acid or a salt thereof to the culture broth in an amount which has an antimicrobial effect on Lactobacillus. Finally, the invention provides use of a C₆-C₁₂ fatty acid or a salt thereof to kill or inhibit the growth of Lactobacillus during recovery, purification, storage or transportation of a liquid enzyme composition.

### DETAILED DESCRIPTION OF THE INVENTION

### Antimicrobial Effect

The liquid composition comprises an enzyme and a C₆-C₁₂ fatty acid or a salt thereof in an amount which has an antimicrobial effect on Lactobacillus, i.e. the amount of the C₆-C₁₂ fatty acid or salt thereof is effective for killing or inhibiting the growth of Lactobacillus.

The antimicrobial effect may be determined by analyzing viable cells (CFU) of the liquid formulation after inoculating with Lactobacillus and incubating for 1-2 weeks at 20-25° C. and comparing with a similar liquid composition without the fatty acid or salt.

### Liquid Composition

The liquid composition generally has a total content of dry matter solids which is above 10% w/w, above 15%, above 20%, above 25%, above 30%, above 35% or above 45%. This may be determined, e.g., by drying at 105° C. until constant weight.

Besides the enzyme, the solids may include one or more of polyols and/or inorganic salts. Suitable polyols include glycerol, propylene glycol (MPG), sorbitol and mono-, di- and tri-saccharides such as glucose, fructose, sucrose and trehalose. The inorganic salt may be an alkali metal halide salt such as NaCl or KCl. The content of polyols and/or inorganic salts may be above 10% w/w, above 15%, above 20%, above 25%, above 30%, above 35% or above 45%.

The liquid composition comprises one or more enzymes. It has an enzyme protein content of at least 1% w/w, particularly at least 2%, at least 5% or at least 10%. The enzyme content may be below 25% w/w, below 20%, below 15% or below 10%.

The liquid composition typically has a pH value of at least 3.5, at least 4.0 or at least 4.5. The pH value is typically below 7, particularly below 6.5, below 6, below 5.5 or below 5. The pH may particularly be in the range 4-5.5.

The liquid composition is typically essentially free from surfactants, both anionic and non-ionic.

### Enzyme

The invention is applicable to liquid formulations comprising one or more enzymes. Examples include enzymes for use in the production of bioethanol from starch or (hemi)cellulosic raw materials (1^{st} and 2^{nd} generation bioethanol) or for use in the textile industry. The enzyme may be a hydrolase, carbohydrase, glycosidase (EC 3.2), alpha-amylase (EC 3.2.1.1), glucan 1,4-alpha-glucosidase (glucoamylase; amyloglucosidase, EC 3.2.1.3), cellulase, hemicellulase, phytase, protease or pullulanase.

Enzymes for Production of Bioethanol from Starch Raw Materials (1G)

The following enzyme products may be formulated according to the invention:

Bacillus alpha-amylases (WO 1996/023873, WO 1999/019467).

Blend of raw-starch hydrolyzing (RSH) alpha-amylase and RSH glucoamylase (WO 2006/069289, WO 2006/069290).

Glucoamylase (WO 1999/028448, WO 2006/069289, WO 2006/069290).

Examples of commercial products are Liquozyme™, Termamyl™ SC and Novozyme BPX, Spirizyme™ (products of Novozymes NS).

Enzymes for Production of Bioethanol from (Hemi)Cellulosic Raw Materials (2G)

The following enzyme products may be formulated according to the invention:

A Trichoderma reesei cellulase preparation containing Aspergillus oryzae betaglucosidase fusion protein (WO 2008/057637) and Thermoascus aurantiacus GH61A polypeptide (WO 2005/074656).

Aspergillus aculeatus GH10 xylanase (WO 94/021785).

A blend of an Aspergillus aculeatus GH10 xylanase (WO 94/021785) and a Trichoderma reesei cellulase preparation containing Aspergillus fumigatus beta-glucosidase (WO 2005/047499) and Thermoascus aurantiacus GH61A polypeptide (WO 2005/074656).

A Trichoderma reesei cellulase preparation containing Aspergillus aculeatus GH10 xylanase (WO 94/021785).

A blend of an Aspergillus fumigatus GH10 xylanase (WO 2006/078256) and Aspergillus fumigatus beta-xylosidase (WO 2011/057140) with a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (PCT/US2011/054185), and Penicillium sp. (emersonii) GH61 polypeptide (WO 2011/041397).

A Trichoderma reesei cellulase preparation containing Aspergillus fumigatus GH10 xylanase (WO 2006/078256) and Aspergillus fumigatus beta-xylosidase (WO 2011/057140).

Examples of commercial products are Cellic® CTec, Cellic HTec, Cellic CTec2, Cellic HTec2, Cellic CTec3, Cellic HTec3 (products of Novozymes NS).

### Alpha-Amylases

The alpha-amylase may be of microbial origin, e.g. bacterial or fungal. The bacterial alpha-amylase may be derived from the genus Bacillus, e.g. from a strain of Bacillus licheniformis, Bacillus amyloliquefaciens, Bacillus subtilis or Bacillus stearothermophilus.

Fungal alpha-amylases include alpha-amylases derived from a strain of the genus Aspergillus, such as, Aspergillus oryzae, Aspergillus niger and Aspergillis kawachii alpha-amylases. Other contemplated wild-type alpha-amylases include those derived from a strain of the genera Rhizomucor and Meripilus, preferably a strain of Rhizomucor pusillus or Meripilus giganteus.

The fungal alpha-amylase may be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

### Glucoamylases

Preferred glucoamylases are of fungal or bacterial origin, e.g. selected from the group consisting of Aspergillus glucoamylases, in particular Aspergillus niger, A. awamori, Aspergillus oryzae

Other glucoamylases include Athelia rolfsii (previously denoted Corticium rolfsii) glucoamylase, Talaromyces glucoamylases, in particular derived from Talaromyces emersonii, Talaromyces leycettanus, Talaromyces duponti, Talaromyces thermophilus.

Bacterial glucoamylases contemplated include glucoamylases from the genus Clostridium, in particular C. thermoamylolyticum and C. thermohydrosulfuricum and Trametes cingulata, Pachykytospora papyracea; and Leucopaxillus giganteus or Peniphora rufomarginata.

### Cellulase

The cellulase may comprise cellobiohydrolases (EC 3.2.1.91), e.g., cellobiohydrolase I and cellobiohydrolase II, as well as endo-glucanases (EC 3.2.1.4) and beta-glucosidases (EC 3.2.1.21). particularly endo-glucahase I and/or II (EG-I, EG-II), cellobiohydrolase I and/or II (CBH-I CBH-II) and/or beta-glucosidase.

The cellulase may be derived from a fungal source, such as a strain of the genus Trichoderma, preferably a strain of Trichoderma reesei; a strain of the genus Humicola, such as a strain of Humicola insolens; or a strain of Chrysosporium, preferably a strain of Chrysosporium lucknowense. The cellulase preparation may comprise a polypeptide having cellulolytic enhancing activity (GH61A).

### Hemicellulase

Preferred hemicellulases include xylanases, arabinofuranosidases, acetyl xylan esterase, feruloyl esterase, glucuronidases, endo-galactanase, mannases, endo or exo arabinases, exo-galactanses and beta-xylosidases.

The xylanase may be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa.

C₆-C₁₂ Fatty Acid or Salt thereof

The C₆-C₁₂ fatty acid may be C₆ (hexanoic acid, caproic acid), C₈ (octanoic acid, caprylic acid) or C₁₀ (decanoic acid, capric acid), particularly C₆ or C₈. Corresponding salts may also be used, particularly Na or K salts.

The content of the C₆-C₁₂ fatty acid or salt in the liquid composition may be at least 0.01% w/w, particularly at least 0.02%, at least 0.05%, at least 0.1%, at least 0.25% or at least 0.5%. The content may be below 1.5% w/w, below 1.0% or below 0.75%.

### Optional Additional Antimicrobial Agent

Optionally, the liquid composition may comprise one or more antimicrobial agents in addition to the C₆-C₁₂ fatty acid or salt. This may serve to improve the inhibition of Lactobacillus growth and/or to inhibit the growth of other microorganisms. Examples are Na or K salts of formate, sorbate, benzoate and sodium sulfite, e.g. in an amount of 0.05-0.8% w/w.

### Killing or Inhibiting Growth of Lactobacillus

According to the invention, a C₆-C₁₂ fatty acid or salt is used to kill or inhibit the growth of Lactobacillus during preparation, recovery, purification, formulation, storage and transportation of a liquid enzyme composition.

The enzyme is typically prepared by cultivation (fermentation) of a suitable microorganism, optionally followed by downstream processing (recovery and/or purification) of the enzyme and formulation into a liquid composition. The enzyme may also be used as a crude product; e.g., the product may be directly obtained from the fermentation broth.

The C₆-C₁₂ fatty acid or salt may be used to inhibit growth of Lactobacillus in the downstream processing of the fermentation broth. After the fermentation process is ended, the enzyme may be recovered from the fermentation broth, using standard technology developed for the enzyme in question.

A process for the recovery of the enzyme from a fermentation broth will typically (but is not limited to) involve one or more of the following steps:
1) pre-treatment of broth (e.g., pH treatment and/or flocculation)
2) removal of cells and other solid material from broth (primary separation)
3) filtration
4) concentration
5) filtration
6) stabilization and standardization.

Apart from the unit operations listed above, a number of other recovery procedures and steps may be applied, e.g., variation in temperature, crystallization, treatment of the solution comprising the compound of interest with active carbon, and use of various adsorbents.

The C₆-C₁₂ fatty acid or salt may be added at any stage of this process in order to inhibit Lactobacillus growth in the subsequent stages.

### EXAMPLES

### Example 1

### Liquid Glucoamylase Formulation with Hexanoate

Two liquid enzyme compositions according to the invention were formulated as follows:
Enzyme: Glucoamylase from Talaromyces
Enzyme content: approx. 19% w/w of enzyme protein
Total solid content: 43% w/w, by addition of glucose
Fatty acid salt: 0.5% w/w of Na-hexanoate
pH: 5.0

### Inhibition of Lactobacillus

Challenge screening was performed by inoculating with 10⁵-10⁶ CFU/ml of Lactobacillus (L. buchneri and L. para paracasei), incubating for two weeks at 20-25° C., and analyzing viable cells (CFU) before and after inoculation and after 1 and 2 weeks.

The results are shown as CFU/ml (detection limit 100):

| | Before in- | After in- | After 1 | After 2 |
|---|---|---|---|---|
| Inhibitor | oculation | oculation | week | weeks |
| Na-hexanoate | ≦100 | 180000 | ≦100 | ≦100 |

Blank tests without the Na-hexanoate show increased CFU after 1 and 2 weeks, both with and without the Na-formate.

Thus, the results demonstrate that the Na-hexanoate in the liquid enzyme formulations has an antimicrobial (germicidal) effect against Lactobacillus.

### Example 2

### Liquid Xylanase and Cellulase Formulation with Hexanoate

A liquid composition with a mixture of two enzymes was formulated as follows:
Enzymes: Xylanase and celllulase
Enzyme content: approx. 18% w/w of enzyme protein
Total solid content: 56.5% w/w, by addition of 22% w/w sucrose
Fatty acid salt: 0.5% w/w of Na-hexanoate
Additional inhibitors: 0.45% w/w Na-benzoate+0.13% w/w K-sorbate
pH: 4.6

The sample was tested as in Example 1. A blank without the Na-hexanoate was included for comparison. Results:

| | Before in- | After in- | | |
|---|---|---|---|---|
| | oculation | oculation | 1 week | 2 weeks |
| Invention | ≦100 | 180000 | ≦100 | ≦100 |
| Blank | ≦100 | 180000 | 82000 | 39000 |

The results demonstrate that the Na-hexanoate in the liquid enzyme formulation has an antimicrobial (germicidal) effect against Lactobacillus.

### Example 3

### Liquid Cellulase and Xylanase Formulation with Hexanoate

A liquid composition with a mixture of two enzymes was formulated as follows:
Enzymes: Cellulase and cellulase
Enzyme content: approx. 18% w/w of enzyme protein
Total solid content: 52% w/w, by addition of 25% w/w sucrose
Fatty acid salt: 0.5% w/w of Na-hexanoate
Additional inhibitors: 0.4% w/w Na-benzoate+0.12% w/w K-sorbate
pH: 4.55

The sample was tested as in Example 1. Results:

| | Before in- | After in- | | |
|---|---|---|---|---|
| | oculation | oculation | 1 week | 2 weeks |
| Invention | ≦100 | 180000 | ≦100 | ≦100 |
| Blank | 200 | 180000 | 60000 | 17000 |

The results demonstrate that the Na-hexanoate in the liquid enzyme formulation has an antimicrobial (germicidal) effect against Lactobacillus.

### Example 4

### Glucoamylase Formulation with Octanoate

A liquid composition with a mixture of two enzymes was formulated as follows:
Enzyme: Glucoamylase from Talaromyces
Enzyme content: approx. 19% w/w of enzyme protein
Total solid content: 43% w/w, by addition of glucose
Fatty acid salt: 0.5% w/w of Na-Caprylate (Na-octanoate)
Additional inhibitor: 0.5% w/w of Na-formate
pH: pH 5.0

The sample was tested as in Example 1. A conventional formulation with 0.75% w/w of Na-formate+0.1% w/w of K-sorbate+0.63% w/w of Na-propionate was included for comparison. Results:

| | Before in- | After in- | After 1 | After 2 |
|---|---|---|---|---|
| Inhibitor(s) | oculation | oculation | week | weeks |
| Invention | ≦100 | 120000 | ≦100 | ≦100 |
| Conventional | ≦100 | 120000 | 940000 | 940000 |

The results demonstrate the strong antimicrobial (germicidal) effect of the Naoctanoate, in comparison with a conventional formulation with formate+sorbate+propionate.

### Example 5

### Comparison with Conventional Inhibitors

A liquid composition with fungal glucoamylases was formulated as follows:
Enzyme: Glucoamylase (mixture of two variants)
Enzyme content: approx. 23% w/w of enzyme protein
Total solid content: 48% w/w, by addition of glucose
Fatty acid salt: 0.3% w/w of Na-Caprylate (Na-octanoate)
Additional inhibitor: with or without 0.3% w/w of Na acetate trihydrate
pH: pH 4.7

The sample was tested as in Example 1. Conventional formulations with sodium benzoate instead of the sodium octanoate were included for comparison. Results:

| | | Before in- | After in- | After 1 | After 2 |
|---|---|---|---|---|---|
| | Inhibitor(s) | oculation | oculation | week | weeks |
| Inven- | Octanoate | ≦100 | 170000 | ≦100 | ≦100 |
| tion | Octanoate + | ≦100 | 170000 | ≦100 | ≦100 |
| | acetate | | | | |
| Conven- | Benzoate | ≦100 | 170000 | 150000 | 120000 |
| tional | Benzoate + | ≦100 | 170000 | 240000 | 80000 |
| | acetate | | | | |

The results demonstrate that the addition of 0.3% w/w of sodium octanoate to a liquid enzyme formulation is adequate to provide a strong antimicrobial (germicidal) effect against Lactobacillus. The addition of acetate as an additional inhibitor was not necessary. The addition of conventional inhibitors (benzoate, acetate) was not effective.

1. A liquid composition, which comprises an enzyme in an amount of at least 1% w/w and a C₆-C₁₂ fatty acid or a salt thereof in an amount which has an antimicrobial effect on Lactobacillus.

2. The composition of claim 1, wherein the fatty acid or salt is hexanoic acid or octanoic acid or a salt thereof.

3. The composition of claim 1, which has a pH value below 7.

4. The composition of claim 1, which has a pH value of at least 3.5, particularly 4-5.5.

5. The composition of claim 1, which is essentially free from a surfactant.

6. The composition of claim 1, which comprises one or more polyols and/or inorganic salts in a total amount of at least 10% w/w.

7. The composition of claim 1, which comprises the enzyme in an amount of at least 2% w/w.

8. The composition of claim 1, wherein the fatty acid or salt thereof is present in an amount of at least 0.01% w/w.

9. The composition of claim 1 which comprises at least 10% w/w of dry matter solids.

10. The composition of claim 1, which comprises a further antimicrobial agent, particularly a formate, a benzoate, a sorbate or a sulfite.

11. A method of recovering, purifying and/or formulating an enzyme from a culture broth, comprising adding a C₆-C₁₂ fatty acid or a salt thereof to the culture broth in an amount which has an antimicrobial effect on Lactobacillus.

12. A method of killing or inhibiting the growth of Lactobacillus during recovery, purification, storage or transportation of a liquid enzyme composition, the method comprising adding a C₆-C₁₂ fatty acid or a salt thereof to the liquid enzyme composition in an amount which has an antimicrobial effect on Lactobacillus.
